(19) United States
(12) Patent Application Publication
(10) Pub. No.: US 2016/0235895 A1
(43) Pub. Date: Aug. 18, 2016

(54) COATED STENT

(71) Applicant: Cook Medical Technologies LLC, Bloomington, IN (US)

(72) Inventor: Kieran Costello, Ballina-Killaloe (IE)

(73) Assignee: Cook Medical Technologies LLC

(21) Appl. No.: 15/007,677

(22) Filed: Feb. 26, 2016

Related U.S. Application Data
(62) Division of application No. 13/656,905, filed on Oct. 22, 2012, now abandoned.
(60) Provisional application No. 61/551,157, filed on Oct. 25, 2011.

(51) Int. Cl. A61L 31/10; B05D 3/04; B05C 13/02

(52) U.S. Cl. CPC A61L 31/10; B05C 13/025; B05D 3/0493; B05D 3/0413; A61L 2420/02

(57) ABSTRACT

An implantable medical device is provided having a plurality of interstices including concave or convex shaped coatings. The concave or convex shaped coatings are configured to straighten and then stretch as the implantable medical device is compressed or elongated, thereby delaying the onset of wrinkling in the coating material. The implantable medical device may include a tubular body having a central body portion and a flange of greater diameter than the central body portion.

### RELATED APPLICATION

This application claims the benefit of priority from U.S. Provisional Application No. 61/551,157, filed Oct. 25, 2011, and titled “Covered Stent”, the contents of which is incorporated herein by reference.

### TECHNICAL FIELD

The present disclosure relates generally to medical devices, and more particularly to coated stents and methods of fabrication thereof.

### BACKGROUND

Stents are tubular shaped medical devices commonly used to maintain patency of diseased body vessels. Stents may be implanted to treat blockages, occlusions, narrowing ailments and other problems that can restrict flow through a vessel. Stents can be implanted, for example, in the coronary and peripheral arteries to maintain blood flow, in the ureters and biliary tract to provide drainage, and in the esophagus to alleviate dysphagia or palliate tumors.

Stents are often delivered in a radially compressed state via a minimally invasive procedure and thereafter expanded to contact and support the inner wall of the targeted vessel. Both self-expanding and balloon-expandable stents are amenable to radial compression and subsequent expansion at the treatment site. Balloon-expandable stents expand in response to the inflation of a balloon, whereas self-expanding stents deploy automatically when released from a delivery device. During expansion, many stents experience longitudinal foreshortening. Conversely, as a stent is compressed it may increase in length. Foreshortening is particularly common with braided stent structures. Braided stents usually comprise one or more helically wound filaments that form a tubular structure having a plurality of closed cells defined by the filament(s). As the stent expands from a compressed state or is compressed, the struts defining the closed cells move relative to one another.

Frequently, such braided stents are provided with a membrane covering over the stent support structure. Self-expanding esophageal stents, for example, are often encased in a silicone membrane to prevent tumor ingrowth and overgrowth, to seal fistulas, and to reduce incidence of tissue perforation. To accommodate the forces experienced during radial compression and expansion of the stent, stent membrane materials are typically made from elastic or flexible materials. Silicone is frequently selected as a stent membrane material due to its biocompatibility, flexibility, availability, and ease of application to the stent structure. Like most elastomers, silicone exhibits a phenomenon known as Poisson's effect. Specifically, as a positive longitudinal strain (ε_{LONG}) is applied to a regularly-shaped silicone article, a corresponding negative transverse strain (ε_{TRANS}) results, tending to cause contraction of the article in a direction perpendicular to the longitudinal strain. The ratio of the transverse strain to the longitudinal strain defines a relationship known as Poisson's ratio. The relationship assumes that the article being stretched can contract with the negative transverse strain, or that the strains are quite low. However, if the ends of the article are constrained and the longitudinal strain exceeds a critical limit (γ_{C}), it has been shown that the material will wrinkle. Specifically, the material will wrinkle near the center of the article but remain sheared near the sites of constraint.

For coated self-expanding braided stents, the elastomeric material in each cell interstice is constrained by the filaments that form the perimeter of the cell. As the stent is elongated or collapsed, the filaments forming the cells move relative to one another and the internal angles of the rhombus shaped cells change. Within the cells, the relationship of the length of the diagonals of the rhombus (i.e., the lines between vertices of a rhombus shaped cell) does not follow a linear relationship as it changes as silicone does to Poisson's ratio. This sets up a conflict of strains between the filament and silicone in each cell. In the case where the stent is elongated (as in the case of stent loading) the elastomer is extended longitudinally, but due to the constraint of the cell perimeter the elastomer wrinkles after the critical strain limit is reached. The wrinkles can form to the external (abluminal) and internal (luminal) sides of the stent. Wrinkles that form to the external aspect of the stent can contact the inner surface of a delivery catheter or sheath that holds the preloaded stent. Elastomers such as silicone can have a relatively high friction coefficient in contact with certain materials. This translates to high loading and deployment forces for stents that contain such wrinkles in the coating. If the forces are high enough this can prevent the stent from being efficiently loaded and deployed.

Optionally, frictional forces can be limited by choosing a larger diameter delivery catheter. This reduces the elongation of the stent in the catheter so that the wrinkling is reduced or does not occur. However, in some cases, it is not be possible to have a through-the-scope device as the delivery catheter diameter may be larger than working channels of available endoscopes. In addition, small diameter delivery devices are advantageous because the device can be placed into tighter strictures and, in some cases, may not necessitate pre-dilation of the targeted lumen. The use of larger diameter delivery sheaths may also be limited because of stricture characteristics.

### SUMMARY

The present disclosure generally provides fully and partially membrane covered stents and methods of fabrication thereof. The stents include at least one cell or interstice with a membrane covering or coating wherein the coating is one of concave or convex in shape. When the stent is in a relaxed state (i.e., fully expanded), the coating within the membrane covered cell is configured to one of a convex or concave shape. As the stent is elongated, during loading into a delivery sheath for example, the coating firstly straightens and eventually begins to stretch. The delay before stretching allows the stent to be elongated further without the onset of wrinkling of the coatings within each cell. The outcome is a lower loading and deployment force during delivery, allowing use of smaller delivery catheters and sheaths. In addition, when the cell coatings are configured to a concave shape (from a perspective view of the abluminal surface), the depressions allow tissue surrounding the stent to ingress between the struts of the cell, thereby anchoring the stent in place.

In one embodiment, an implantable medical device comprises a tubular body extending longitudinally between a proximal end and a distal end. The tubular body includes a plurality of interstices defined by one or more structural elements (e.g., filament or wire) forming the tubular body. Each interstice includes a luminal side and an abluminal side. A coating material occupies a plurality of the interstices. The coatings may be concave or convex in shape. In other words, the coatings may extend inward toward the lumen of the tubular body, or extend outward away from the lumen of the tubular body. The coatings in the interstices are configured to approach planarity as the tubular body is compressed or elongated.

In certain embodiments, the interstices may be arranged into annular rows along a longitudinal axis of the implantable medical device. In certain embodiments, the implantable medical device may comprise a central body portion and at least one flange having a greater diameter than the central body portion. The flange may comprise a plurality of interstices having concave or convex shaped coatings or coverings. In certain embodiments, the central body portion may comprise a plurality of interstices having substantially planar shaped coatings when the device is in a relaxed, fully expanded state.

In another aspect, a method of fabricating a stent having concave shaped cell coatings is provided. The method includes applying a coating material to a closed-cell stent structure. A pressure differential may be created between an abluminal and a luminal surface of the stent. In certain embodiments, the luminal surface may be subjected to a lower pressure than the abluminal surface. This pressure differential is configured to draw in the coating material occupying the closed cells of the stent structure so as to form concave shaped cell coatings upon curing. In an alternative embodiment, the luminal surface may be subjected to a higher pressure than the abluminal surface to provide convex shaped cell coatings. In certain embodiments, the stent may be rotated about its central longitudinal axis as the pressure differential is applied and the coatings cure within the closed cells of the stent structure. In certain embodiments, the proximal and distal openings of the stent structure may be sealed to facilitate creation of the pressure differential. In another embodiment, the pressure differential may be created by applying positive pressure to an abluminal surface of the stent using a device configured to blow air (e.g., an air blower or a heat blade).

In another aspect, a mandrel is provided for use in fabrication of stents comprising concave shaped cell coverings. The mandrel includes a solid cylindrical body extending longitudinally from a proximal end to a distal end. The cylindrical body includes an external surface where a plurality of wells are located. The wells may be configured to receive a coating material therein during the fabrication of a stent comprising concave shaped cell coverings. In certain embodiments, the mandrel includes a central body portion and a flange of greater diameter than the central body portion. The flange comprises a plurality of the wells. In certain embodiments, the central body portion of the mandrel may lack a plurality of the wells.

In another aspect, a method is provided for forming a stent having concave shaped cell coatings. The method includes placing a closed-cell stent structure on a mandrel comprising a solid cylindrical body extending longitudinally from a proximal end to a distal end. The cylindrical body includes an external surface and a plurality of wells located at the external surface. The wells are configured to receive a coating material therein. The stent may be adjusted so that the stent interstices align with the wells. A coating material may be applied to the mounted stent such that the coating material at least partially fills the plurality of wells. The coating material may be at least partially cured with the stent mounted on the mandrel. Upon reaching a desired level of cure, the stent may be removed from the mandrel.

Other devices, systems, methods, features and advantages will be, or will become, apparent to one with skill in the art upon examination of the following figures and detailed description. It is intended that all such additional devices, systems, methods, features and advantages be included within this description, and be protected by the following claims.

### BRIEF DESCRIPTION OF THE DRAWINGS

The embodiments will be further described in connection with the attached drawing figures. It is intended that the drawings included as a part of this specification be illustrative of the exemplary embodiments and should in no way be considered as a limitation on the scope of the invention. Indeed, the present disclosure specifically contemplates other embodiments not illustrated but intended to be included in the claims. Moreover, it is understood that the figures are not necessarily drawn to scale.

FIG. 1 illustrates a covered stent.

FIG. 2 illustrates a cross-sectional view of the covered stent of FIG. 1.

FIG. 3 illustrates a cross-sectional view of the covered stent of FIG. 1.

FIG. 4 illustrates a method of forming a covered stent

FIG. 5 illustrates a method of forming a covered stent.

FIGS. 6A-6C illustrate a method of forming a covered stent.

FIGS. 7-8 illustrate a mandrel for preparing a covered stent.

FIGS. 9-10 illustrate a covered stent implanted in a body lumen.

FIG. 10A illustrates a covered stent implanted in a body lumen.

### DETAILED DESCRIPTION

The exemplary embodiments illustrated provide the discovery of methods and apparatuses for manufacturing covered stents that allow for greater elongation and a corresponding greater reduction in diameter without the onset of wrinkling of the elastomeric coating so as to reduce loading and deployment forces and reduce the diameter of the delivery device. The present invention is not limited to those embodiments described herein, but rather, the disclosure includes all equivalents including those of different shapes, sizes, and configurations, including but not limited to, other types of stents. The devices and methods can be used in any field benefiting from a stent. Additionally, the devices and methods are not limited to being used with human beings, others are contemplated, including but not limited to, animals.

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The present disclosure also contemplates other embodiments “comprising,” “consisting of” and “consisting essentially of,” the embodiments or elements presented herein, whether explicitly set forth or not.

The term “proximal,” as used herein, refers to a direction that is generally towards a physician during a medical procedure.

The term “distal,” as used herein, refers to a direction that is generally towards a target site within a patient's anatomy during a medical procedure.

The term “biocompatible,” as used herein, refers to a material that is substantially non-toxic in the in vivo environment of its intended use, and that is not substantially rejected by the patient's physiological system. A biocompatible structure or material, when introduced into a majority of patients, will not cause an undesirably adverse, long-lived or escalating biological reaction or response. Such a response is distinguished from a mild, transient inflammation which typically accompanies surgery or implantation of foreign objects into a living organism.

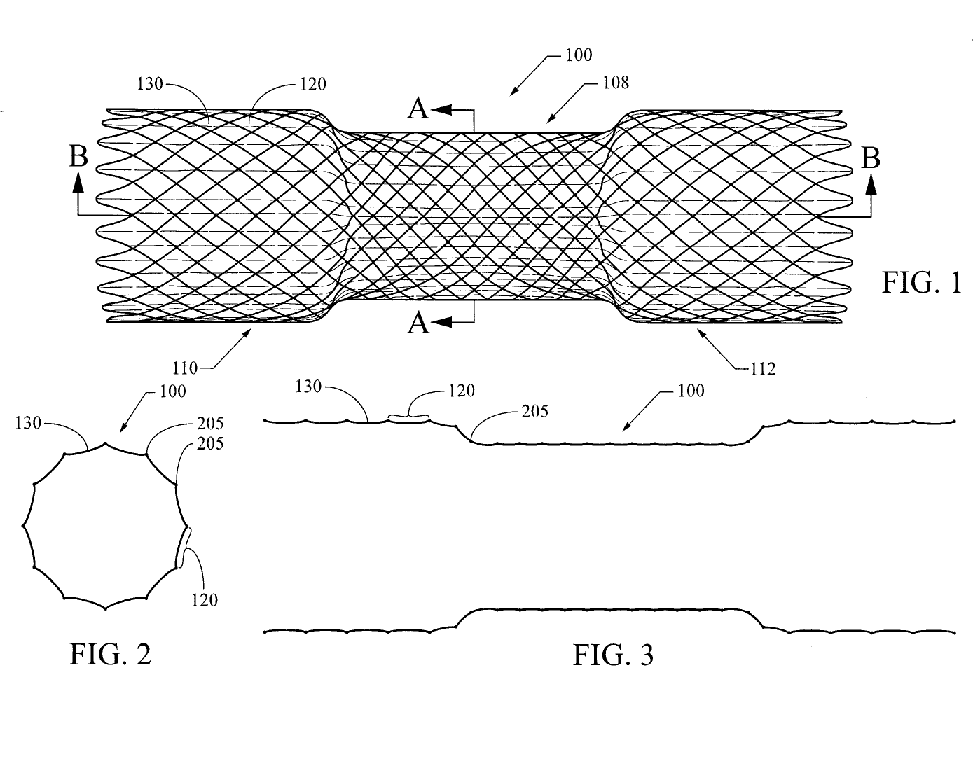

A more detailed description of the embodiments will now be given with reference to FIGS. 1-10A. Throughout the disclosure, like reference numerals and letters refer to like elements. The present disclosure is not limited to the embodiments illustrated; to the contrary, the present disclosure specifically contemplates other embodiments not illustrated but intended to be included in the claims.

FIG. 1 illustrates a self-expanding stent 100 having a framework or structure comprised of one or more helically wound or braided filaments. Intersections of filament in the braid pattern create a plurality of rhombus shaped cells 120 defined at their perimeter by the filament(s). The framework includes a tubular shaped central body portion 108 extending longitudinally between two flanges 110 and 112. A membrane material covers the stent support structure, with each cell 120 having a membrane covering 130 occupying the cell interstice. As will be described in greater detail below, when the stent is in a relaxed (i.e., fully expanded) state, the coating in each cell is concave in shape and extends inward in a direction toward the luminal space of the stent. As the stent is elongated during compression (e.g., during loading into a delivery device) and the cells increase in length along the longitudinal axis of the stent, the concave shaped coatings in each cell firstly straighten and eventually begin to stretch. The delay before stretching allows the stent to be extended further without the onset of wrinkling of the membrane material in each cell. This results in a lower loading and deployment force when the stent is delivered from a compressed state to the target site. Accordingly, smaller diameter delivery devices may be used to deliver the stent.

FIG. 2 illustrates a cross-sectional view of stent 100 from line A-A toward flange 110. As shown, each cell 120 has a concave shaped membrane covering 130 that extends inward toward the luminal space of the stent. Vertices of the rhombus shaped cells are depicted by points 205. FIG. 3 illustrates another cross-sectional view of stent 100 along line B-B (i.e., along the longitudinal axis of the stent). As shown, the cells have concaved shaped membrane coverings 130 along the entire length of the stent. However, other coating configurations are possible. For example, the concaved shaped cell coatings may be limited to the flanges of the stent, the central body portion of the stent, and combinations thereof. The distribution pattern of the concaved shaped cell coatings may also be controlled. There may be combinations of conventionally coated cells, concaved shaped coatings, convex shaped coatings, and cells lacking a membrane covering, for example.

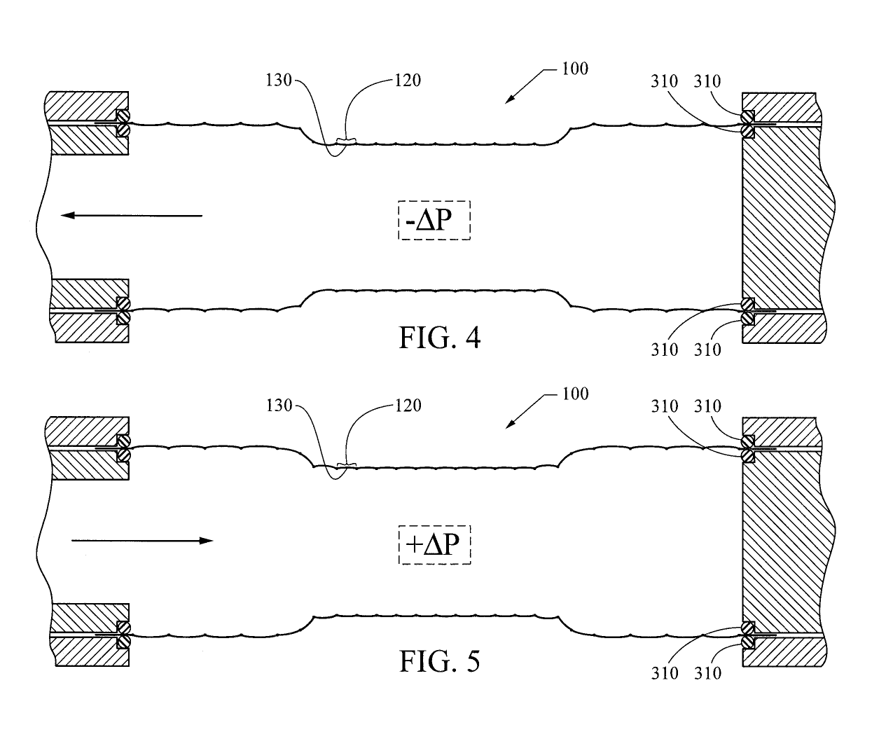

FIG. 4 illustrates a method of forming a covered stent having concave shaped cell coverings. The figure shows a slice of stent 100 along line B-B. In this exemplary embodiment, a coating material may firstly be applied to stent 100. The stent may be secured to a mandrel or other securing device. The ends of the stent (i.e., the proximal and distal openings of the luminal space of the stent) may then be closed by seals 310. Next, using a vacuum source, a reduced pressure environment may be created in the luminal space of the stent. For example, a vacuum may be applied by piercing one of the seals 310 with a needle attached to a vacuum line by way of a luer lock. The reduced pressure environment may cause the coatings in each cell to take a concave shape during the curing process. The desired level of concavity may be adjusted by controlling the composition of the coating material, the magnitude of the applied vacuum, the time of vacuum application, and the level of curing allowed before applying the vacuum.

In certain embodiments, a covered stent may be fabricated having one or more convex shaped cell coatings. In other words, cell coatings may be prepared that bow outward toward the abluminal surface of the stent support structure. FIG. 5 illustrates a method of forming a covered stent 100 with convex shaped cell coverings. FIG. 5 shows a slice of stent 100 along line B-B (see FIG. 1) where the cells 120 have a convex shaped membrane covering 130. Similar to the procedure as described with respect to FIG. 4, a coating material may be applied to the stent and the stent may be secured to a mandrel or other securing device. The ends of the stent may be sealed with seals 310 so that a positive pressure can be applied to the luminal space of the stent. The positive pressure may be applied as the coatings cure within each cell interstice, the positive pressure causing the coatings to bow outward during the cure.

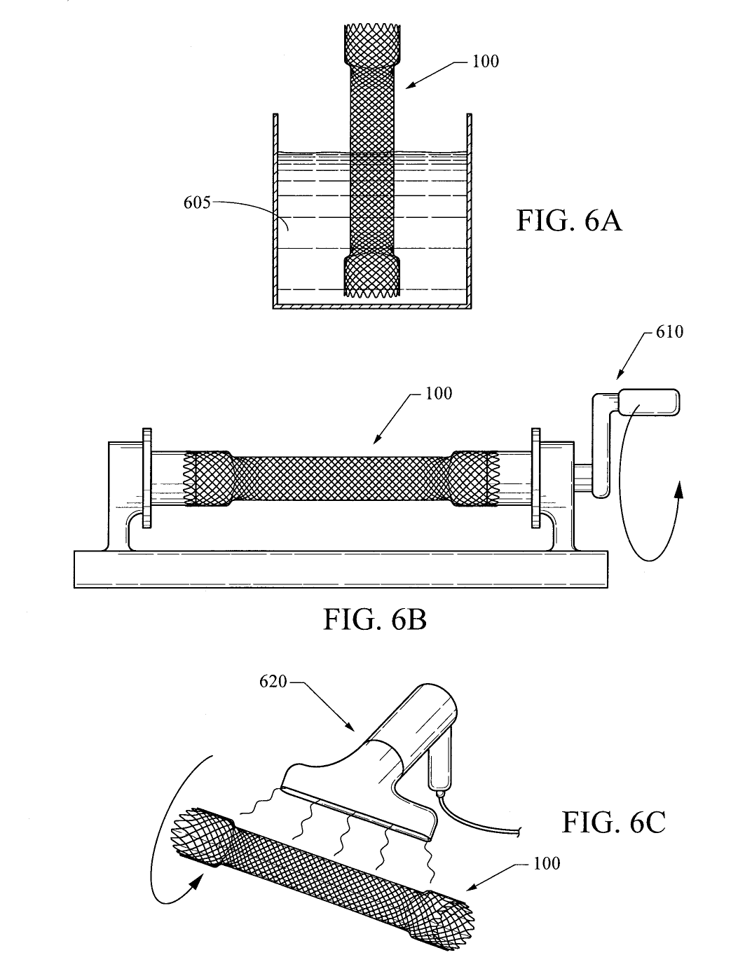

FIGS. 6A-6C illustrate an alternative method of forming covered stents having concave shaped cell coatings. Stent 100 may be dipped in a coating material 605, such as silicone for example, and thereafter secured to a rotation device 610. As stent 100 is rotated and the coating material cures, a source of air 620, optionally heated air, may be applied to the abluminal surface of the stent, thereby causing the cell coatings to uniformly bow inward toward the stent luminal space. The desired level of concavity can be selected based on the coating material, the intensity of air pressure applied to the stent abluminal surface, whether the air is heated or cooled, the time of application of air to the stent, and the level of curing allowed prior to application of the air source.

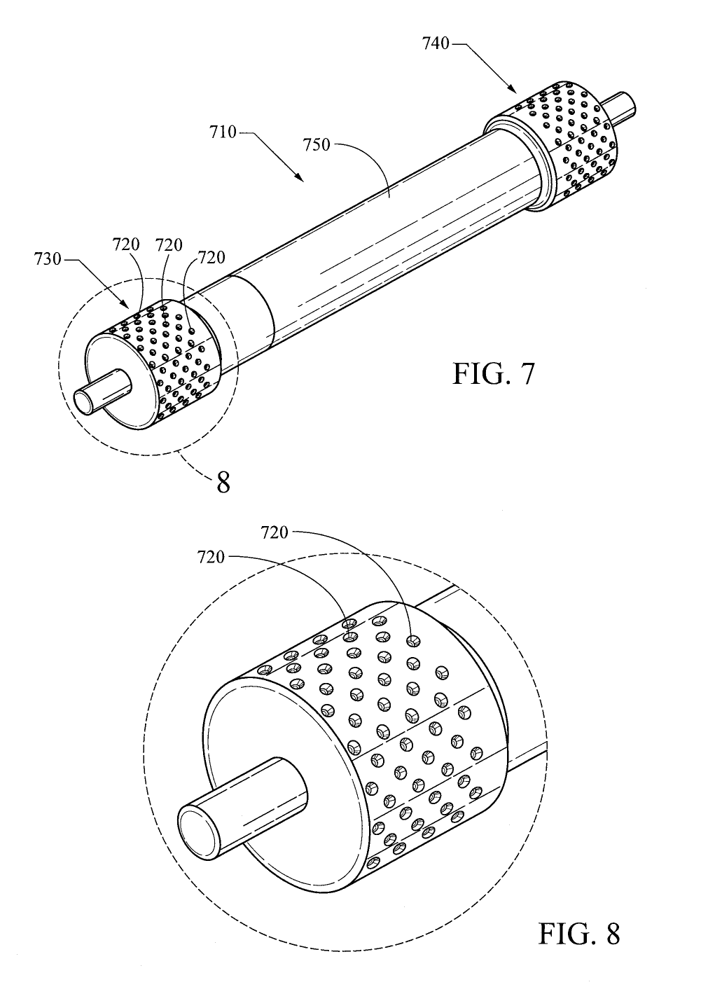

FIGS. 7 and 8 illustrate a mandrel 710 that may be used to form covered stents having concave shaped cell coatings. The mandrel includes a plurality of wells 720 configured to be aligned with the cell interstices of stent 100. As is shown, the wells may be limited to the flanges 730 and 740 of the mandrel. However, any distribution pattern can be selected based on the desired configuration and distribution of concaved shaped cell coatings in the finished stent. Thus, in certain embodiments, the mandrel may include wells on the central body portion 750. Using mandrel 710, a covered stent having concave shaped cell coatings may be formed by securing the uncovered stent to the mandrel, and thereafter applying the coating material thereto, by for example, dipping or spraying. Upon curing of the coating material, the stent may be removed from the mandrel to provide a covered stent having concave shaped cell coatings. The thickness and degree of concavity of the cell coatings can be controlled, at least partially, by the adjusting the size, shape, and depth of the wells. For example, larger diameter wells may provide a larger diameter cell covering, or deeper wells may be used to provide cell coverings of greater concavity. In another example, the wells could be oval in shape to balance the longitudinal strain (ε_{LONG}) with the transverse strain (ε_{TRANS}) and minimize the onset of wrinkling.

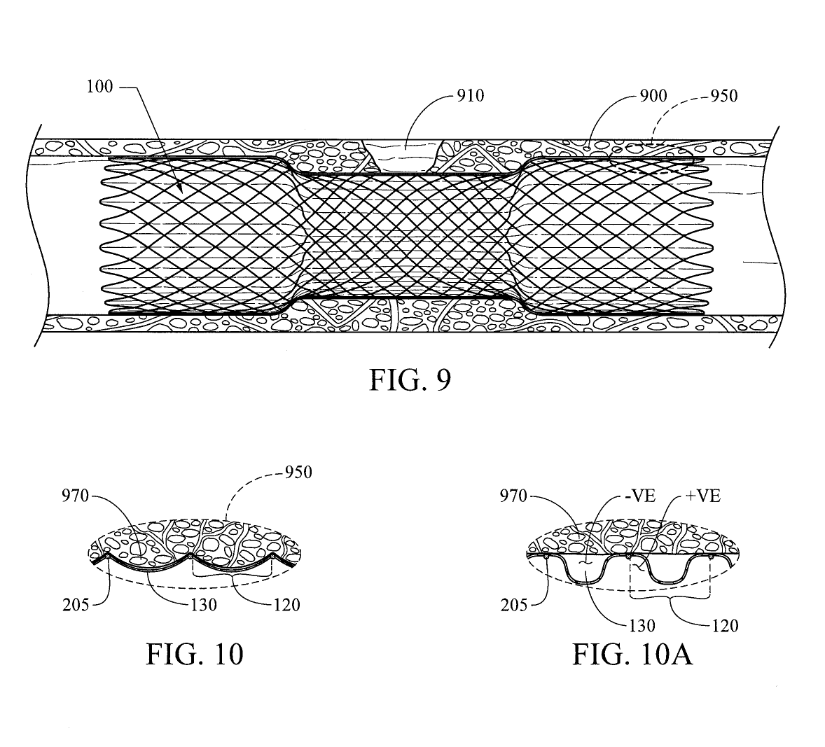

FIGS. 9 and 10 illustrate stent 100 implanted in body lumen 900 in order to prevent the body lumen from closing due to stricture 910. FIG. 10 shows an enhanced view of area 950 where stent 100 engages a portion of the body lumen. The stent allows a degree of tissue ingress into the cell interstices because the cell coatings extend inward toward the stent lumen. Specifically, tissue 970 can ingress into the interstices of cells 120 up to the point of contacting concave shaped membranes 130. This limited ingress may be beneficial to secure the stent at the site of implantation and reduce the incidence of stent migration.

FIG. 10A illustrates the concave shapes in the membrane acting as a suction cup on the tissue. If air is displaced from the abluminal side of the stent (for example, by food passing in the stent lumen), a relative negative pressure can exist in the space between the stent and tissue 970. This relative negative pressure provides suction between the stent and the tissue, reducing the incidence of stent migration. Other shapes in the membrane acting as a suction cup on the tissue are contemplated including, but not limited to, oval shapes and circular shapes.

A stent according to the present disclosure may have any suitable braid angle. The radial force of the stent may be controlled by adjusting the braid angle accordingly. Stents with higher braid angles typically exert greater radial force and exhibit greater foreshortening during expansion from a compressed state. Stents with lower braid angles typically exert lower radial force and experience less foreshortening upon expansion. In some instances, the stent braid angle can be lowered because the membrane covering typically adds rigidity to the stent structure. In addition to adjusting the braid angle, the radial force of the stent can be adjusted through selection of particular filament materials, as well as the shape and size of the filaments or wires forming the stent structure.

Although the illustrated embodiments illustrate a stent having a central body portion and two flanges, other stent configurations are possible. For example, a stent may include a single flange, two asymmetrically shaped flanges, or may entirely lack flanges and instead have a uniform or substantially uniform diameter along the entire length of the stent. A stent may include a uniform diameter along the length of the stent but include slightly flared proximal and/or distal ends. The central body portion may smoothly transition to a flange or flare, or alternatively, may progressively step up in diameter to a flange or flare. Generally, a stent may be implanted in a vessel (e.g., esophagus, duodenum, colon, trachea, or the like) such that the central body portion engages a diseased area and the flanges or ends engage healthy tissue adjacent the diseased area. Preferably, the flanges are configured to anchor the stent at the site of implantation, thereby reducing the incidence of antegrade and retrograde migration. Preferably, the flanges are sized and shaped to accommodate the vessel or organ of implantation. For example, stents destined for lower esophageal implantation may have differently shaped and sized flanges compared to a stent designed for upper esophageal implantation. In certain embodiments, the flanges may include features or configurations designed to reduce incidence of tissue perforation and overgrowth. For example, the ends (e.g, the crown cells) of the flanges may curve or bend inward toward the stent lumen to minimize tissue damage at or near the stent ends. In certain embodiments, a stent may include other design elements configured to secure the stent at the site of implantation. For example, in certain embodiments, a stent may include small anchors, clips, hooks, or barbs that will anchor the stent to the internal wall of the targeted body lumen. In other embodiments, the stent may be sutured to the site of implantation at one or more portions of the stent structure.

A stent may include one or more components configured to aid in visualization and/or adjustment of the stent during implantation, repositioning, or retrieval. For example, a stent may include one or more radiopaque markers configured to provide for fluoroscopic visualization for accurate deployment and positioning. Radiopaque markers may be affixed (e.g., by welding, gluing, suturing, or the like) at or near the ends of the stent at a cross point of filament(s) in the braid pattern. In certain embodiments, a stent may include four radiopaque markers with two markers affixed to a first flange and two to a second flange. Optionally, radiopacity can be added to a stent through coating processes such as sputtering, plating, or co-drawing gold or similar heavy metals onto the stent. Radiopacity can also be included by alloy addition. Radiopaque materials and markers may be comprised of any suitable biocompatible materials, such as tungsten, tantalum, molybdenum, platinum, gold, zirconium oxide, barium salt, bismuth salt, hafnium, and/or bismuth subcarbonate.

A stent may include one or more loops, lassos, or sutures on the stent structure to facilitate repositioning or removal of the stent during or after implantation. For example, a stent may include a loop at or near the proximal end of the stent. The loop material may circumscribe the flange and in certain embodiments may be wound through the absolute end cells to affix the loop to the stent. The loop may comprise any appropriate biocompatible materials, such as for example, suture materials or other polymeric or metallic materials such as polyethylene, ultra-high molecular weight polyethylene, polyester, nylon, stainless steel, nitinol, or the like. Optionally, the lasso may be coated with a material, such as polytetrafluoroethylene, to reduce frictional interactions of the lasso with surrounding tissue.

Stents of the present disclosure may be self-expanding, mechanically expandable, or a combination thereof. Self-expanding stents may be self-expanding under their inherent resilience or may be heat activated wherein the stent self-expands upon reaching a predetermined temperature or range of temperatures. One advantage of self-expanding stents is that traumas from external sources or natural changes in the shape of a body lumen do not permanently deform the stent. Thus, self-expanding stents may be preferred for use in vessels that are subject to changes in shape and/or changes in position, such as those of the peripheral and gastrointestinal systems. Peripheral vessels regularly change shape as the vessels experience trauma from external sources (e.g, impacts to arms, legs, etc.); and many gastrointestinal vessels naturally change shape as peristaltic motion advances food through the digestive tract.

One common procedure for implanting a self-expanding stent involves a two-step process. First, if necessary, the diseased vessel may be dilated with a balloon or other device. The stent may be loaded within a sheath that retains the stent in a compressed state for delivery to the targeted vessel. The stent may then be guided to the target anatomy via a delivery catheter and thereafter released by retracting or removing the retaining sheath. Once released from the sheath, the stent may radially expand until it contacts and presses against the vessel wall. In some procedures, self-expanding stents may be delivered with the assistance of an endoscope and/or a fluoroscope. An endoscope provides visualization as well as working channels through which devices and instruments may be delivered to the site of implantation. A fluoroscope also provides visualization of the patient anatomy to aid in placement of an implantable device, particularly in the gastrointestinal system.

Mechanically expandable stents (e.g., balloon expandable stents) may be made from plastically deformable materials (e.g., 316L stainless steel). A balloon-expandable stent may be crimped and delivered in a reduced diameter and thereafter expanded to a precise expanded diameter. Balloon expandable stents can be used to treat stenosed coronary arteries, among other vessels. One common procedure for implanting a balloon expandable stent involves mounting the stent circumferentially on a balloon-tipped catheter and threading the catheter through a vessel passageway to the target area. Once the balloon is positioned at the targeted area, the balloon may be inflated to dilate the vessel and radially expand the stent. The balloon may then be deflated and removed from the passageway.

Expandable stents according to the present disclosure may be formed by any suitable method as is known in the art. In certain embodiments, the expandable stents may be fabricated by braiding, weaving, knitting, crocheting, welding, suturing, or otherwise machining together one or more filaments or wires into a tubular frame. Such stents may be referred to as braided, woven, or mesh stents. A braided stent may be fabricated by, for example, use of a braiding mandrel having specifically designed features (e.g., grooves and detents) for creating such a stent. A variety of braiding patterns are possible, such as for example, one-under and one-over patterns or two-under and two-over patterns. The filaments or wires may be of various cross-sectional shapes. For example, the filaments or wires may be flat in shape or may have a circular-shaped cross-section. The filaments or wires may have any suitable diameter, such as for example, from about 0.10 to about 0.30 mm As will be described in greater detail below, the expandable stents may be formed from a variety of biocompatible materials. For example, the filaments or wires may comprise one or more elastically deformable materials such as shape memory alloys (e.g., 304 stainless steel, nitinol, and the like).

Alternatively, the expandable stents may be formed from metallic or polymeric sheets or tubular blanks. For example, a stent framework comprising a selected pattern of struts defining a plurality of cells or interstices may be fabricated by subjecting a metallic or polymeric sheet or tubular blank to laser cutting, chemical etching, high-pressure water etching, mechanical cutting, cold stamping, and/or electro discharge machining. After obtaining a sheet of cut, etched or machined material with the appropriate strut pattern, the sheet may be rolled into a tubular shape to form the stent framework. The stent framework may also be machined from a tubular blank, thereby eliminating the need for a rolling step.

A stent may be made from any suitable biocompatible material(s). For example, a stent may include materials such as stainless steel, nitinol, MP35N, gold, tantalum, platinum or platinum iridium, niobium, tungsten, Iconel® (available from Special Metals Corporation, Huntington, W.Va.), ceramic, nickel, titanium, stainless steel/titanium composite, cobalt, chromium, cobalt/chromium alloys, magnesium, aluminum, or other biocompatible metals and or composites or alloys. Examples of other materials that may be used to form stents include carbon or carbon fiber; cellulose acetate, cellulose nitrate, silicone, polyethylene terephthalate, polyurethane, polyamide, polyester, polyorthoester, polyanhydride, polyether sulfone, polycarbonate, polypropylene, ultra high molecular weight polyethylene, polytetrafluoroethylene, or another biocompatible polymeric material, or mixtures or copolymers of these; polylactic acid, polyglycolic acid or copolymers thereof; a polyanhydride, polycaprolactone, polyhydroxybutyrate valerate or another biodegradable polymer, or mixtures or copolymers of these; a protein, an extracellular matrix component, collagen, fibrin, or another biologic agent; or a suitable mixture of any of these.

A stent may be fabricated to any suitable dimensions. A stent having a particular length and diameter may be selected based on the targeted vessel. For example, a stent designed for esophageal implantation may have a length ranging from about 5 cm to about 15 cm and a body diameter of about 15 mm to about 25 mm. Optionally, an esophageal stent may include one or more flanges or flares of about 10 mm to about 25 mm in length and about 20 mm to about 30 mm in diameter. A stent designed for colon implantation may have a length ranging from about 5 cm to about 15 cm and a body diameter of about 20 mm to about 25 mm Optionally, a colonic stent may include one or more flanges having a diameter of about 25 mm to about 35 mm.

In certain embodiments, a stent according to the present disclosure includes membrane covering over the entire stent framework from the proximal end to the distal end. In other embodiments, the stent may have covering over a central portion of the structure but have uncovered ends or flanges. Any suitable biocompatible material may be used as the membrane covering. Preferably, the membrane covering is an elastic or flexible material that can adapt to radial compression of a stent prior to delivery, as well as foreshortening of a stent during expansion from a compressed state. Suitable membrane materials include, for example, silicones (e.g. polysiloxanes and substituted polysiloxanes), polyurethanes, thermoplastic elastomers, polyolefin elastomers, polyethylene, polytetrafluoroethylene, nylon, and combinations thereof In one preferred embodiment, the membrane covering comprises silicone. In certain embodiments, where the stent will be implanted at or near an acidic environment (e.g., will be exposed to gastric fluids), preferably the membrane covering is resistant to acid degradation.

The membrane covering may be applied to a stent by any suitable method as is known in the art. For example, the membrane may be applied by spraying, dipping, painting, brushing, or padding. Generally, the membrane covering or coating has a thickness ranging from about 0.0025 mm to about 2.5 mm, from about 0.01 mm to about 0.5 mm, or from about 0.03 mm to about 0.07 mm. The thickness of the membrane may be selected, for example, by controlling the number of dips or passes made during the application process.

In certain embodiments, a stent may include one or more bioactive agents coated on the stent surfaces. A bioactive agent may be applied directly on the surface of the stent (or on a primer layer which is placed directly on the surface of the stent). Alternatively, the bioactive agent may be mixed with a carrier material and this mixture applied to the stent. In such configuration, the release of the bioactive agent may be dependent on factors including composition, structure and thickness of the carrier material. The carrier material may contain pre-existing channels, through which the bioactive agent may diffuse, or channels created by the release of bioactive agent, or another soluble substance, from the carrier material.

One or more barrier layers may be deposited over the layer containing the bioactive agent. A combination of one or more layers of bioactive agent, mixtures of carrier material/bioactive, and barrier layers may be present. The bioactive agent may be mixed with a carrier material and coated onto the stent and then over coated with barrier layer(s). Multiple layers of bioactive agent, or mixtures of carrier material/bioactive, separated by barrier layers may be present to form a multicoated stent. Different bioactive agents may be present in the different layers.

The carrier material and/or the barrier layer can include a bioelastomer, PLGA, PLA, PEG, Zein, or a hydrogel. In certain other embodiments, the carrier material and/or the barrier layer includes microcrystalline cellulose, hydroxypropylmethyl cellulose, hydroxypropyl cellulose, a cellulose product, a cellulose derivative, a polysaccharide or a polysaccharide derivative. The carrier material and/or barrier layer may include lactose, dextrose, mannitol, a derivative of lactose, dextrose, mannitol, starch or a starch derivative. The carrier material and/or barrier layer may include a biostable or a biodegradable material, for example, a biostable or biodegradable polymer.

A variety of bioactive agents may be applied to the stent in accordance with the intended use. For example, bioactive agents that may be applied include antiproliferative/antimitotic agents including natural products such as vinca alkaloids (vinblastine, vincristine, and vinorelbine), paclitaxel, rapamycin analogs, epidipodophyllotoxins (etoposide, teniposide), antibiotics (dactinomycin (actinomycin D) daunorubicin, doxorubicin and idarubicin), anthracyclines, mitoxantrone, bleomycins, plicamycin (mithramycin) and mitomycin, enzymes (for example, L-asparaginase which systemically metabolizes L-asparagine and deprives cells which do not have the capacity to synthesize their own asparagine); antiplatelet agents such as (GP) II b/IIIa inhibitors and vitronectin receptor antagonists; antiproliferative/antimitotic alkylating agents such as nitrogen mustards (mechlorethamine, cyclophosphamide and analogs, melphalan, chlorambucil), ethylenimines and methylmelamines (hexamethylmelamine and thiotepa), alkyl sulfonates-busulfan, nirtosoureas (carmustine (BCNU) and analogs, streptozocin), trazenes-dacarbazinine (DTIC); antiproliferative/antimitotic antimetabolites such as folic acid analogs (methotrexate), pyrimidine analogs (fluorouracil, floxuridine, and cytarabine), purine analogs and related inhibitors (mercaptopurine, thioguanine, pentostatin and 2-chlorodeoxyadenosine fcladribinel); platinum coordination complexes (cisplatin, carboplatin), procarbazine, hydroxyurea, mitotane, aminoglutethimide; hormones (i.e. estrogen); anticoagulants (heparin, synthetic heparin salts and other inhibitors of thrombin); fibrinolytic agents (such as tissue plasminogen activator, streptokinase and urokinase), aspirin, dipyridamole, ticlopidine, clopidogrel, abciximab; antimigratory; antisecretory (breveldin); anti-inflammatory: such as adrenocortical steroids (cortisol, cortisone, fludrocortisone, prednisone, prednisolone, 6α-methylprednisolone, triamcinolone, betamethasone, and dexamethasone), non-steroidal agents (salicylic acid derivatives i.e. aspirin; para-aminophenol derivatives i.e. acetaminophen; indole and indene acetic acids (indomethacin, sulindac, and etodalac), heteroaryl acetic acids (tolmetin, diclofenac, and ketorolac), arylpropionic acids (ibuprofen and derivatives), anthranilic acids (mefenamic acid, and meclofenamic acid), enolic acids (piroxicam, tenoxicam, phenylbutazone, and oxyphenthatrazone), nabumetone, gold compounds (auranofin, aurothioglucose, gold sodium thiomalate); immunosuppressives (cyclosporine, tacrolimus (FK-506), sirolimus (rapamycin), tacrolimus, everolimus, azathioprine, mycophenolate mofetil); angiogenic agents: vascular endothelial growth factor (VEGF), fibroblast growth factor (FGF); angiotensin receptor blockers; nitric oxide and nitric oxide donors; anti-sense oligionucleotides and combinations thereof; cell cycle inhibitors, mTOR inhibitors, and growth factor receptor signal transduction kinase inhibitors; retenoids; cyclin/CDK inhibitors; endothelial progenitor cells (EPC); angiopeptin; pimecrolimus; angiopeptin; HMG co-enzyme reductase inhibitors (statins); metalloproteinase inhibitors (batimastat); protease inhibitors; antibodies, such as EPC cell marker targets, CD34, CD133, and AC 133/CD133; Liposomal Biphosphate Compounds (BPs), Chlodronate, Alendronate, Oxygen Free Radical scavengers such as Tempamine and PEA/NO preserver compounds, and an inhibitor of matrix metalloproteinases, MMPI, such as Batimastat.

A bioactive agent may be applied, for example, by spraying, dipping, pouring, pumping, brushing, wiping, vacuum deposition, vapor deposition, plasma deposition, electrostatic deposition, ultrasonic deposition, epitaxial growth, electrochemical deposition or any other method known to the skilled artisan.

Prior to applying a membrane covering, and/or a bioactive agent, a stent may be polished, cleaned, and/or primed as is known in the art. A stent may be polished, for example, with an abrasive or by electropolishing. A stent may be cleaned by inserting the stent into various solvents, degreasers and cleansers to remove any debris, residues, or unwanted materials from the stent surfaces. Optionally, a primer coating may be applied to the stent prior to application of a membrane covering, bioactive, or other coating. Preferably, the primer coating is dried to eliminate or remove any volatile components. Excess liquid may be blown off prior to drying the primer coating, which may be done at room temperature or at elevated temperatures under dry nitrogen or other suitable environments including an environment of reduced pressure. A primer layer may comprise, for example, silane, acrylate polymer/copolymer, acrylate carboxyl and/or hydroxyl copolymer, polyvinylpyrrolidone/vinylacetate copolymer (PVP/VA), olefin acrylic acid copolymer, ethylene acrylic acid copolymer, epoxy polymer, polyethylene glycol, polyethylene oxide, polyvinylpyridine copolymers, polyamide polymers/copolymers polyimide polymers/copolymers, ethylene vinylacetate copolymer and/or polyether sulfones.

A stent according to the present disclosure may be delivered to a body lumen using various techniques. Generally, under the aid of endoscopic and/or fluoroscopic visualization a delivery device containing the stent is advanced into the vicinity of the target anatomy. The targeted lumen may be predilated with a balloon catheter or other dilation device, if necessary. Preferably, the stent is delivered in a compressed state in a low profile delivery device. This approach may reduce the risk of tissue perforations during delivery. Once the delivery device is in place, the stent may be released from the retaining sheath or the like. In one preferred embodiment, a stent may be delivered with a controlled release system (e.g., Evolution™ Controlled-Release Stent, Cook Endoscopy Inc., Winston-Salem, N.C.). A controlled release device permits the physician to slowly release the stent from the retaining sheath and in some instances, recapture the stent to allow for repositioning. After implantation, the delivery device and any other devices (e.g., wire guides, catheters, etc.) may be removed.

While various embodiments of the presently disclosed stents having concave or convex shaped cell coatings have been described, it will be apparent to those of ordinary skill in the art that many more embodiments and implementations are possible within the scope of the present disclosure. Accordingly, the disclosure is not to be restricted except in light of the attached claims and their equivalents.

From the foregoing, the discovery of methods and apparatuses for manufacturing covered stents that allow for greater elongation and a corresponding greater reduction in diameter without the onset of wrinkling of the elastomeric coating so as to reduce loading and deployment forces and reduce the diameter of the delivery device will likely improve stent procedures. It can be seen that the embodiments illustrated and equivalents thereof as well as the methods of manufacturer may utilize machines or other resources, such as human beings, thereby reducing the time, labor, and resources required to manufacturer the embodiments. Indeed, the discovery is not limited to the embodiments illustrated herein, and the principles and methods illustrated herein can be applied and configured to any stent and equivalents.

Those of skill in the art will appreciate that embodiments not expressly illustrated herein may be practiced within the scope of the present discovery, including that features described herein for different embodiments may be combined with each other and/or with currently-known or future-developed technologies while remaining within the scope of the claims presented here. It is therefore intended that the foregoing detailed description be regarded as illustrative rather than limiting. It is understood that the following claims, including all equivalents, are intended to define the spirit and scope of this discovery. Furthermore, the advantages described above are not necessarily the only advantages of the discovery, and it is not necessarily expected that all of the described advantages will be achieved with every embodiment of the discovery.

1-8. (canceled)

9. A method of forming a stent comprising concave shaped cell coatings, comprising:
applying a coating material to a closed-cell stent structure; and
creating a pressure differential between an abluminal surface of the stent structure and a luminal surface of the stent structure, the luminal surface subjected to a lower pressure than the abluminal surface, the pressure differential configured to draw in the coating material occupying closed cells of the stent structure so as to form concave shaped cell coatings.

10. The method of claim 9 wherein the stent is rotated about a central longitudinal axis of the stent as the pressure differential is applied and the coatings cure within the closed cells of the stent structure.

11. The method of claim 9 wherein the stent comprises a proximal opening and a distal opening, wherein the proximal opening and distal opening are sealed as the pressure differential is applied to the stent structure.

12. The method of claim 10 wherein the pressure differential is created by applying positive pressure to the abluminal surface using a device configured to blow air.

13. The method of claim 12 wherein the air is heated air.

14. A mandrel configured for use in fabrication of stents comprising concave shaped cell coverings, the mandrel comprising:
a solid cylindrical body extending longitudinally from a proximal end to a distal end, the solid cylindrical body comprising an external surface; and
a plurality of wells located at the external surface, the wells configured to receive a coating material therein during the fabrication of a stent comprising concave shaped cell coverings.

15. The mandrel of claim 14 further comprising a central body portion and a flange, wherein the flange comprises a plurality of the wells.

16. The mandrel of claim 15 wherein the central body portion comprises a substantially uniform external surface.

17. The mandrel of claim 15 wherein the mandrel comprises a first flange and a second flange, each flange comprising a plurality of the wells.

18. The mandrel of claim 14, wherein the mandrel comprises a first longitudinal portion and a second longitudinal portion, the first longitudinal portion configured to mate with the second longitudinal portion.

19. A method of forming a stent comprising concave shaped cell coatings, comprising:
placing a closed-cell stent structure on a mandrel comprising a solid cylindrical body extending longitudinally from a proximal end to a distal end, the solid cylindrical body comprising an external surface and a plurality of wells located at the external surface, the wells configured to receive a coating material therein;
applying a coating material to the stent and the mandrel such that the coating material partially fills the plurality of wells; and
at least partially curing the coating material on the stent and mandrel.

20. The method of claim 19, wherein the mandrel further comprises a central body portion disposed between the proximal end and the distal end and a flange, wherein the flange comprises a plurality of the wells.

21. A method of forming an implantable medical device, comprising:
providing a braided tubular body extending between a proximal end and a distal end, said tubular body comprising a plurality of rhombus shaped interstices defined by one or more helically wound structural elements forming the braided tubular body, each of the plurality of rhombus shaped interstices comprising a luminal side and an abluminal side;
extending an uncured coating material between the one or more helically wound structural elements so as to occupy at least one of the plurality of rhombus shaped interstices;
forming one of a concave or convex shape in a luminal side of the uncured coating material, and further forming the other of the concave or convex shape in an abluminal side of the uncured coating material; and
curing the uncured coating material.

22. The method of claim 21 wherein the braided tubular body is movable between a compressed configuration and an expanded configuration, and the step of providing the braided tubular body includes disposing the braided tubular body in the expanded configuration.

23. The method of claim 21 wherein the step of forming the uncured coating material includes creating a pressure differential between the luminal and abluminal sides of the uncured coating material so as to form the concave or convex shape.

24. The method of claim 21 wherein the step of forming the uncured coating material includes engaging one of the luminal or abluminal sides of the uncured coating material with a mandrel having either projections or depressions disposed in the surface thereof.
